(19) United States
(12) Patent Application Publication
(10) Pub. No.: US 2006/0100579 A1
(43) Pub. Date: May 11, 2006

(54) Apparatus and methods for controlling pressurization of a body cavity

(75) Inventors: Tracy D. Maahs, Rancho Santa Margarita, CA (US); Rebecca S. Inderbitzen, San Diego, CA (US); Vahid Saadat, Saratoga, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: USGI Medical Inc., San Clemente, CA (US)

(21) Appl. No.: 10/898,548

(22) Filed: Jul. 23, 2004

(51) Int. Cl. A61M 1/00; A61M 31/00

(52) U.S. Cl. 604/151; 604/506

(57) ABSTRACT

Apparatus and methods are provided for controlling pressurization of a body cavity by controlling the rate and magnitude of pressurization during insufflation and/or exsufflation. In one variation, a pump may be used to either draw suction or to pressurize a body cavity. The pump may be connected to a controller that controls one or more regulators and/or valves for regulating pressurization. The controller preferably is programmable to allow a medical practitioner to input and/or rapidly alter desired pressurization parameters. In another variation, one or more pumps may be provided to simultaneously and independently control pressurization of multiple body cavities, such as a patient's stomach and peritoneal cavity.

### BACKGROUND OF THE INVENTION

### Field of the Invention

The present invention relates to methods and apparatus for controlling pressurization of a body cavity. More particularly, the present invention provides methods and apparatus for controlling the rate and magnitude of pressurization during insufflation and/or exsufflation.

Extreme or morbid obesity is a serious medical condition pervasive in the United States and other countries. Its complications include hypertension, diabetes, coronary artery disease, stroke, congestive heart failure, multiple orthopaedic problems and pulmonary insufficiency with markedly decreased life expectancy.

Applicant has previously described methods and apparatus for endoscopically and/or laparoscopically/transgastrically treating morbid obesity, for example, in U.S. patent application Ser. No. 10/734,562, filed Dec. 12, 2003; Ser. No. ______ [Attorney Docket No. 02149-000800US], filed May 7, 2004; Ser. No. ______ [Attorney Docket No. 02149-001400US], filed May 7, 2004; and Ser. No. ______ [Attorney Docket No. 02149-001300US], filed May 10, 2004, all of which are incorporated herein by reference in their entireties. The techniques described therein may require control over the pressurization of a working space created within a patient's stomach, which may be achieved by insufflating the stomach.

Known insufflators typically allow insufflation pressure to be regulated, but may not provide for control of insufflation rate. Furthermore, exsufflation often is achieved by utilizing a pressure differential between the insufflated space and the ambient environment. For example, a tube may be placed down a patient's throat or through a laparoscopic port into the patient's stomach, such that a pressure differential between the patient's stomach and the ambient environment may induce exsufflation of the stomach. Exsufflation may also be achieved via suction; however, the suction often is drawn at an uncontrolled rate dictated by the strength of the suction pump. Thus, exsufflation may occur at a substantially uncontrolled rate subject to the magnitude of the pressure differential or to the strength of suction. Furthermore, control of the magnitude of pressurization during exsufflation often is poor.

In view of the foregoing, it would be desirable to provide methods and apparatus for controlling pressurization of a body cavity, which provide control over a magnitude of pressurization.

It would also be desirable to provide methods and apparatus that provide control over a rate of pressurization.

It would be desirable to provide methods and apparatus that facilitate rapid changes in a degree of pressurization.

It would be desirable to provide methods and apparatus for simultaneously and independently controlling pressurization of multiple body cavities.

### BRIEF SUMMARY OF THE INVENTION

Controlled pressurization of one or more body cavities is achieved utilizing apparatus and methods described herein. Specifically, methods and apparatus for controlling the rate and magnitude of pressurization during insufflation and/or exsufflation are described.

In one variation, a pump may be used to either draw suction or to pressurize a body cavity. The pump may be connected to a controller that controls one or more regulators and/or valves for regulating pressurization. A flow sensor may be connected to the controller in a feedback control loop to facilitate control of the rate of insufflation or exsufflation. Likewise, a pressure sensor may be connected to the controller in a feedback control loop to maintain a desired magnitude of pressurization. The controller preferably is programmable via a switch or other user input, such as a foot pedal or a dial, that allows a medical practitioner to input and/or rapidly alter desired pressurization parameters. In this manner, it is expected that significant changes in cavity pressurization may be achieved in an expedited fashion. Furthermore, use of a foot pedal may free up a medical practitioner's hands for performance of other tasks.

In another variation, one or more pumps may be provided to simultaneously and independently control pressurization of multiple body cavities, such as a patient's stomach and peritoneal cavity. Pressurization may be achieved endoluminally, transluminally, laparoscopically, or via any other known technique.

Methods of using apparatus of the present invention also are provided.

### BRIEF DESCRIPTION OF THE DRAWINGS

FIG. 1 is a schematic view of apparatus for controlling pressurization of a body cavity.

FIG. 2 is a schematic view of an exemplary interface for a controller and/or user input of the apparatus.

FIG. 3 is a schematic graph illustrating an exemplary relationship between the rate and magnitude of pressurization.

FIG. 4 is a schematic view of a variation of the apparatus for simultaneously and independently controlling pressurization of multiple body cavities.

FIG. 5 is a schematic view of another variation of the apparatus for simultaneously and independently controlling pressurization of multiple body cavities.

### DETAILED DESCRIPTION OF THE INVENTION

Controlled pressurization of one or more body cavities is achieved utilizing apparatus and methods described herein. Specifically, methods and apparatus for controlling the rate and magnitude of pressurization during insufflation and/or exsufflation are described.

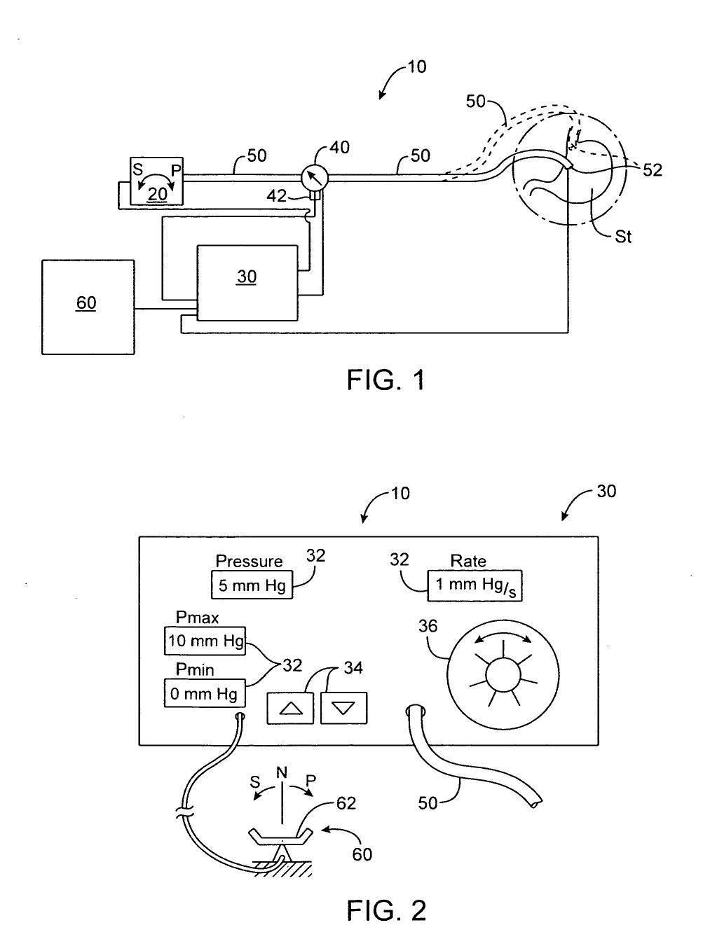

With reference to FIG. 1, a first variation of the apparatus is described. Apparatus 10 comprises pump 20 that may be run in either direction to provide either suction S or compressed air pressurization P. Multiple pumps alternatively may be provided for separately controlling suction and pressurization. Pump 20 is electrically connected to controller 30 and is fluidly connected to regulator valve 40, e.g., via tubing 50. Regulator valve 40 is also connected to controller 30, which controls the valve to achieve desired pressurization parameters. A medical practitioner programs the desired parameters into the controller via user input 60. The user input may comprise, for example, dials, switches, foot pedals, graphical user interfaces or any other known user input. An illustrative interface arrangement is described hereinbelow with respect to FIG. 2.

As seen in FIG. 1, valve 40 may comprise sensor 42, e.g., a flow sensor, that is connected to controller 30 in a feedback loop to facilitate regular monitoring and/or correction of flow through the valve. In this manner, a rate of pressurization or suction through tubing 50 may be controlled. Likewise, tubing 50 may comprise a sensor, such as pressure sensor 52 connected at or near a distal end of the tubing, e.g., on an exterior surface of the tubing. Sensor 52 may be connected to controller 30 in a feedback loop to facilitate regular monitoring and/or correction of a magnitude of pressurization within a body cavity, thereby providing automated control over the magnitude.

Tubing 50 extends beyond valve 40 for placement within a body cavity. The tubing illustratively is shown advanced transgastrically into a patient's stomach St for controlling pressurization of the stomach. The tubing also is shown in dotted profile to illustrate an alternative per-oral advancement down the patient's throat into the stomach.

A user may input desired pressurization parameters via input 60, and controller 30 achieves those parameters by controlling pump 20 and/or regulator valve 40 subject to flow and pressure readings from sensors 42 and 52, respectively. Apparatus 10 thereby provides for controlled pressurization of a body cavity. Furthermore, the parameters of insufflation and/or exsufflation may be changed rapidly subject to altered inputs from the user.

In FIG. 1, both pump 20 and regulator valve 40 are controlled through controller 30; however, it should be understood that either the pump or the valve alternatively may be controlled directly by a medical practitioner. It is expected that the control provided by apparatus 10, as well as the rapid ability to insufflate and/or exsufflate a space, will facilitate challenging medical procedures. These may include, but are not limited to, procedures within a patient's stomach, such as endoluminal or transluminal gastric reduction or restriction.

Referring now to FIG. 2, an exemplary interface for controller 30 and user input 60 is described. As shown, controller 30 may comprise one or more displays 32 that provide information to the user, such as the rate of insufflation or exsufflation, the magnitude of pressurization, etc. Furthermore, the controller may comprise one or more buttons 34 or dials 36 for programming desired pressurization parameters, such that user input 60 and controller 30 optionally may be (at least partially) integrated into a single unit.

User input 60 illustratively is shown as foot pedal 62 connected to controller 30. The pedal comprises neutral position N where, for example, a magnitude of pressurization may be maintained at its current level. The pedal pivots about the central position, and movement, e.g., clockwise, instructs the controller to increase pressurization P or insufflate a body cavity. The rate of increase may be determined by the degree to which the foot pedal is advanced clockwise past neutral position N. Likewise, counterclockwise movement may decrease a rate of insufflation until the neutral point is again achieved. Further counterclockwise movement may draw suction S to reduce pressurization, i.e., to exsufflate the body cavity at an increasing rate.

In FIG. 2, tubing 50 passes through controller 30; and valve 40, as well as sensor 42, pump 20, etc., may, for example, be integrated into the controller. Additional interfaces for controller 30 and/or user input 60 will be apparent. Controller 30 preferably allows for input of maximum and/or minimum pressurization magnitudes in order to reduce a risk of injury to a patient.

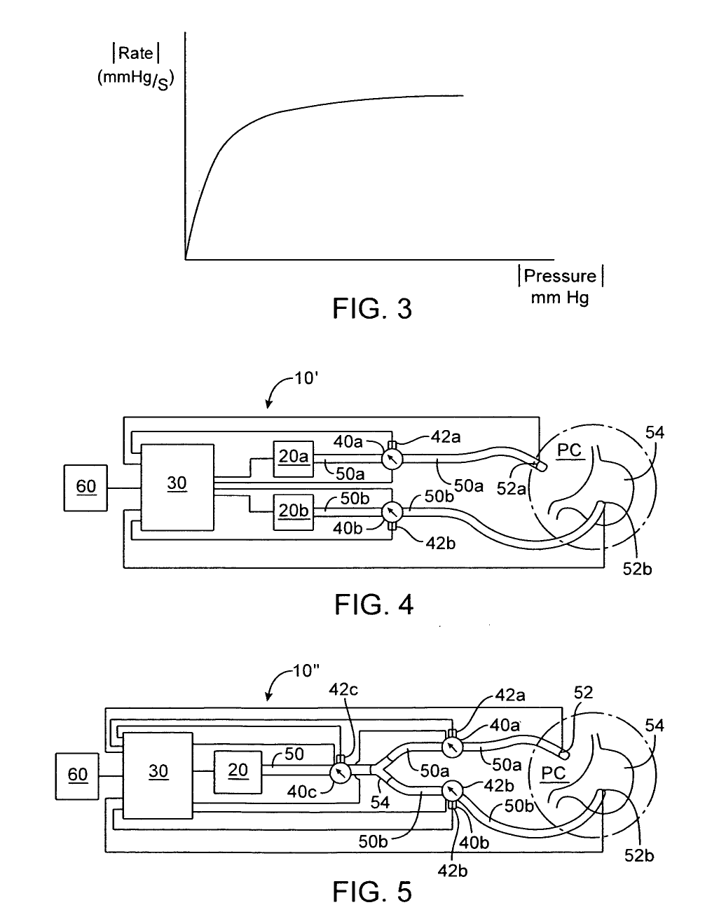

Pressurization magnitude and rate optionally may be related to one another in a variety of ways. For example, the parameters may be directly proportional. Alternatively, the parameters may be inversely proportional. As yet another alternative, the parameters may be mutually independent. FIG. 3 illustrates a logarithmic relationship between absolute magnitude and rate of pressurization. Additional and alternative relationships will be apparent.

Referring now to FIG. 4, a variation of apparatus 10 is described that facilitates simultaneous and independent control of pressurization of multiple body cavities. Apparatus 10′ comprises first and second pumps 20a and 20b that are connected via tubings 50a and 50b to first and second regulator valves 40a and 40b, respectively. The pumps also preferably are coupled to controller 30. The outlets of tubings 50a and 50b may be placed in separate body cavities in order to simultaneously and independently provide controlled pressurization of two different body cavities. As will be apparent, additional apparatus may be provided for controlling pressurization of more than two body cavities.

In FIG. 4, tubing 50b is again disposed within stomach St for controlling pressurization of the gastric lumen, while tubing 50a illustratively is disposed within peritoneal cavity PC. In this configuration, pressurization of the stomach and the peritoneal cavity may be achieved simultaneously and independently. Pressurization of any other body cavities may be similarly controlled.

FIG. 5 illustrates another variation 10″ of the apparatus, whereby simultaneous and independently controlled pressurization of multiple body cavities is achieved with a single pump. In FIG. 5, tubing 50 extends from pump 20 before splitting at, e.g., Y-connector 54 into tubings 50a and 50b. Tubing 50a is coupled to regulator valve 40a, while tubing 50b is coupled to regulator valve 40b. The pump and the regulators are connected to controller 30 for simultaneous and independently controlled pressurization of multiple body cavities.

Optionally, regulator valve 40c and flow sensor 42c also may be provided between pump 20 and Y-connector 54, such that a pressure differential between the body cavities pressurized via tubings 50a and 50b may be utilized to drive insufflation and desufflation. Thus, pressurization control of one body cavity may be either independent or dependent on pressurization of another body cavity. Additional variations will be apparent.

Although preferred illustrative embodiments of the present invention are described hereinabove, it will be apparent to those skilled in the art that various changes and modifications may be made thereto without departing from the invention. It is intended in the appended claims to cover all such changes and modifications that fall within the true spirit and scope of the invention.

What is claimed is:

1. Apparatus for controlling pressurization of a body cavity, the apparatus comprising:
a pump configured to draw suction and compress air;
tubing fluidly coupled to the pump and configured for disposal within a body cavity;
a regulator valve coupled to the tubing; and
a programmable controller configured to control a magnitude and rate of pressurization of the body cavity via the apparatus.

2. The apparatus of claim 1, wherein the apparatus comprises a flow sensor to facilitate control of the rate of pressurization of the body cavity.

3. The apparatus of claim 1, wherein the apparatus comprises a pressure sensor to facilitate control of the magnitude of pressurization.

4. The apparatus of claim 1, wherein the apparatus comprises a user input for programming the controller.

5. The apparatus of claim 4, wherein the user input is chosen from the group consisting of switches, dials, graphical user interfaces, foot pedals, and combinations thereof.

6. The apparatus of claim 4 further comprising a display that provides current pressurization parameters.

7. The apparatus of claim 1, wherein the apparatus comprises separate pumps for drawing suction and compressing air.

8. The apparatus of claim 1, wherein the apparatus is configured to simultaneously control pressurization of multiple body cavities.

9. The apparatus of claim 8, wherein the apparatus is configured to independently control pressurization of the multiple body cavities.

10. The apparatus of claim 9, wherein the apparatus comprises separate regulator valves for each body cavity.

11. A method of controlling pressurization of a body cavity, the method comprising:
providing apparatus comprising a pump configured to draw suction and compress air, tubing fluidly coupled to the pump, and a programmable controller;
inserting an outlet of the tubing into the body cavity;
programming the controller with desired pressurization parameters; and
controlling pressurization of the body cavity by controlling the pump with the controller.

12. The method of claim 11, wherein programming the controller further comprises programming the controller through a user input.

13. The method of claim 11, wherein controlling pressurization further comprises controlling a magnitude and rate of pressurization.

14. The method of claim 11, wherein controlling pressurization further comprises measuring at least one pressurization parameter.

15. The method of claim 14 further comprising feeding the at least one pressurization parameter back into the controller.

16. The method of claim 14, wherein measuring at least one pressurization parameter further comprises measuring a rate or magnitude of pressurization.

17. The method of claim 11, wherein controlling pressurization further comprises controlling a magnitude and rate of pressurization through a regulator valve coupled to the tubing.

18. The method of claim 11, wherein controlling pressurization further comprises controlling pressurization of multiple body cavities.

19. The method of claim 18, wherein controlling pressurization of multiple body cavities further comprises simultaneously and independently controlling pressurization of the multiple body cavities.

20. The method of claim 18, wherein controlling pressurization of multiple body cavities further comprises controlling pressurization of a patient's stomach and peritoneal cavity.

21. The method of claim 11, wherein controlling pressurization of the body cavity further comprises controlling pressurization of a patient's stomach.
